(19) United States
(12) Patent Application Publication
(10) Pub. No.: US 2024/0237976 A1
(43) Pub. Date: Jul. 18, 2024

(54) STERILE ADAPTER ASSEMBLIES FOR ROBOTIC SURGICAL SYSTEMS

(71) Applicant: Covidien LP, Mansfield, MA (US)

(72) Inventors: Jon Cooke, Cambridge (GB); Matthew M. Marinovich, London (GB); Lewis Butler, Leeds (GB); Antony R. Burness, Cambridge (GB)

(21) Appl. No.: 18/561,666

(22) PCT Filed: Jul. 13, 2022
(86) PCT No.: PCT/CA2022/051092

§ 371 Date: Nov. 16, 2023

Related U.S. Application Data
(60) Provisional application No. 63/222,154, filed on Jul. 15, 2021.

(51) Int. Cl. A61B 17/00; A61B 34/30

(52) U.S. Cl. CPC A61B 17/00234; A61B 34/30; A61B 2017/00398; A61B 2017/00486; A61B 2034/302

(57) ABSTRACT

A surgical system includes a motor assembly and an adapter assembly. The motor assembly includes a motor coupler and the adapter assembly supports an adapter coupler that engages with the motor coupler. When the adapter and motor couplers are misaligned, the motor coupler can rotate relative to the adapter coupler to align the motor and adapter couplers so they can rotate together.

### CROSS REFERENCE TO RELATED APPLICATIONS

This application is a 371 National Stage Application of International Application No. PCT/CA2022/051092, filed Jul. 13, 2022, which claims the benefit of, and priority to, U.S. Provisional Patent Application No. 63/222,154, filed on Jul. 15, 2021, the entire contents of each of which are hereby incorporated herein by reference.

### TECHNICAL FIELD

This disclosure relates to robotic systems and, more particularly, to sterile adapter assemblies for robotic surgical systems.

### BACKGROUND

Robotic surgical systems include control drive assemblies supporting surgical instruments used in laparoscopic and/or robotic surgery. These surgical instruments are operably coupled to a control drive unit of the robotic surgical system by a sterile adapter assembly. The sterile adapter assembly enables drive force from motors of the control drive unit to be imparted to distal end effectors of the surgical instruments. These motors are remotely controlled by a clinician to enable the surgical instruments to robotically perform a surgical task within a body cavity of a patient, and often in remote locations within the body cavity that are not easily accessed without robotic surgical systems.

### SUMMARY

According to an aspect of this disclosure, a robotic surgical system includes a motor block module and a sterile adapter assembly. The motor block module supports a motor assembly. The motor assembly includes a motor having a shaft assembly. The shaft assembly includes a rotatable shaft and a motor coupler assembly secured to the rotatable shaft. The motor coupler assembly includes a motor coupler and a spring engaged with the motor coupler. The sterile adapter assembly is mounted to the motor block module and supports an adapter coupler that is positioned to engage with the motor coupler. The adapter coupler is positioned to impart axial force onto the motor coupler sufficient to compress the spring when the adapter coupler and the motor coupler are rotationally misaligned. The motor coupler is rotatable relative to the adapter coupler to rotationally align the motor coupler and the adapter coupler. The spring is positioned to move the motor coupler toward the adapter coupler when the motor coupler and the adapter coupler are rotationally aligned such that the adapter coupler and the motor coupler can rotate together.

In aspects, the motor coupler assembly may include a collar assembly that secures to the rotatable shaft. The spring may extend between the motor coupler and the collar assembly. The spring may be supported on a fastener that secures the motor coupler to the collar assembly. The motor coupler may be axially movable along the fastener and relative to the collar assembly as the spring moves between a compressed position and an expanded position.

In aspects, the sterile adapter assembly may include an adapter body and an adapter plate that support the adapter coupler therebetween. The adapter body may include an anti-rotation feature that selectively engages with an anti-rotation feature of the adapter coupler to prevent the adapter coupler from rotating when the motor coupler and the adapter coupler are misaligned.

In aspects, the sterile adapter assembly may include a tongue and the motor block module may support a distal end cap. The distal end cap may define a locking groove that is positioned to receive the tongue of the sterile adapter assembly to secure the sterile adapter assembly to the distal end cap.

In aspects, the motor coupler may include a drive channel on a distal end portion thereof and the adapter coupler may include a proximal cleat that is positioned to be received within the drive channel of the motor coupler when the adapter coupler and the motor coupler are rotationally aligned. The adapter coupler may include a distal cleat that is configured to engage with an instrument coupler of a surgical instrument.

According to one aspect, this disclosure is directed to a surgical system. The surgical system includes a motor block module, an adapter assembly, and a surgical instrument. The motor block module supports a motor assembly. The motor assembly includes a motor having a shaft assembly. The shaft assembly includes a shaft and a motor coupler assembly secured to the shaft. The motor coupler assembly includes a motor coupler and a spring engaged with the motor coupler. The adapter assembly is mounted to the motor block module and supports an adapter coupler that is positioned to engage with the motor coupler. The adapter coupler is positioned to impart force onto the motor coupler sufficient to compress the spring when the adapter coupler and the motor coupler are misaligned. The motor coupler is movable relative to the adapter coupler to align the motor coupler and the adapter coupler. The spring is positioned to move the motor coupler toward the adapter coupler when the motor coupler and the adapter coupler are aligned such that the adapter coupler and the motor coupler can move together. The surgical instrument is securable to the adapter assembly.

According to yet another aspect, this disclosure is directed to a robotic surgical system including a motor block module, an adapter assembly, and a surgical instrument. The motor block module supports a motor assembly including a motor having a shaft assembly. The shaft assembly includes a shaft and a motor coupler secured to the shaft. The adapter assembly is mountable to the motor block module and supports an adapter coupler that is positioned to engage with the motor coupler. The motor coupler is rotatable relative to the adapter coupler when the motor coupler and the adapter coupler are misaligned and rotatable with the adapter coupler when the motor coupler and the adapter coupler are aligned. The surgical instrument is securable to the adapter assembly and includes an instrument coupler that is positioned to engage with the adapter coupler. The adapter coupler is positioned to rotate relative to the instrument coupler when the instrument coupler and the adapter coupler are rotationally misaligned to rotationally align the adapter coupler with the instrument coupler.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIGS. 2-4 are progressive views illustrating surgical instruments of the robotic surgical system of FIG. 1 being manipulated within a body cavity of the patient;

FIG. 5 is an enlarged, perspective view of the robotic surgical system of FIG. 1 and illustrating a control drive assembly thereof being used to perform a surgical procedure on a patient;

FIG. 6 is a perspective view of a motor block module of the control drive assembly shown attached to a surgical instrument of the control drive assembly by a sterile adapter assembly;

FIG. 7 is an enlarged perspective view, with parts separated, of a distal end portion of the motor block module of FIG. 6, the sterile adapter assembly of FIG. 6, and a proximal end portion of the surgical instrument of FIG. 6;

FIG. 8 is an enlarged, perspective view illustrating the sterile adapter assembly of FIG. 7 secured to the distal end portion of the motor block module of FIG. 7 and separate from the surgical instrument of FIG. 7;

FIG. 9 is an enlarged, perspective view of the sterile adapter assembly of FIG. 6;

FIG. 10 is a perspective view of FIG. 9 with parts separated;

FIG. 11 is another perspective view of FIG. 8 with portions thereof shown in phantom for clarity;

FIG. 12 is an enlarged, perspective view illustrating drive structure of the motor block module and the sterile adapter assembly of FIG. 6;

FIG. 13 is a perspective view of a motor assembly of the motor block module of FIG. 6;

FIG. 14 is a perspective view, with parts separated of the motor assembly of FIG. 13; and

FIGS. 15-19 are progressive views illustrating drive structure of the motor block module, the adapter assembly, and the surgical instrument of FIG. 6 being coupled together.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user, while the term “proximal” refers to that portion of structure, closer to the user. As used herein, the term “clinician” refers to a doctor, nurse, or other care provider and may include support personnel and/or equipment operators.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments.

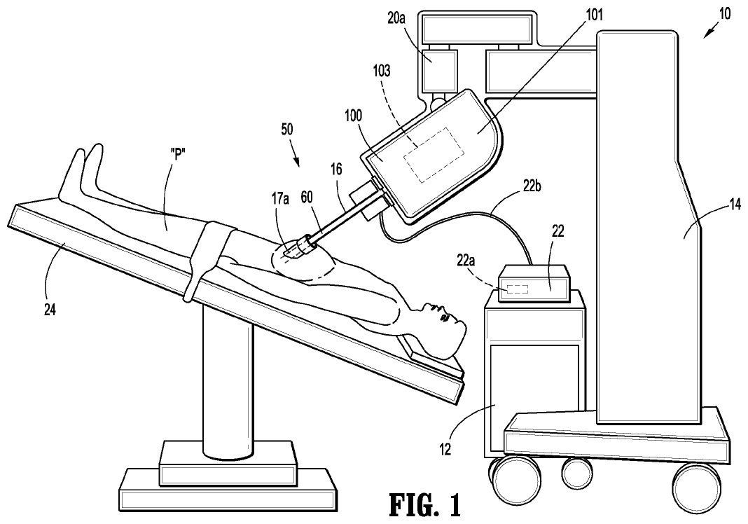

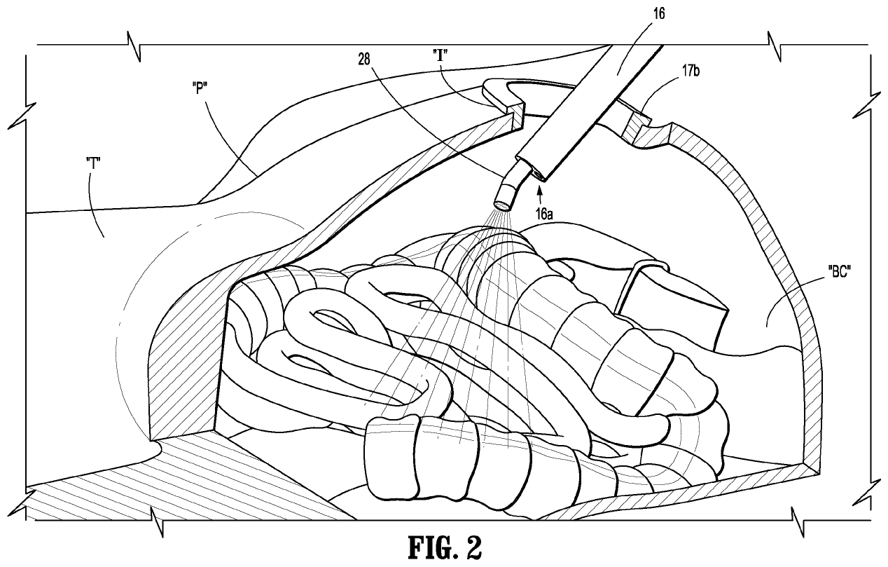

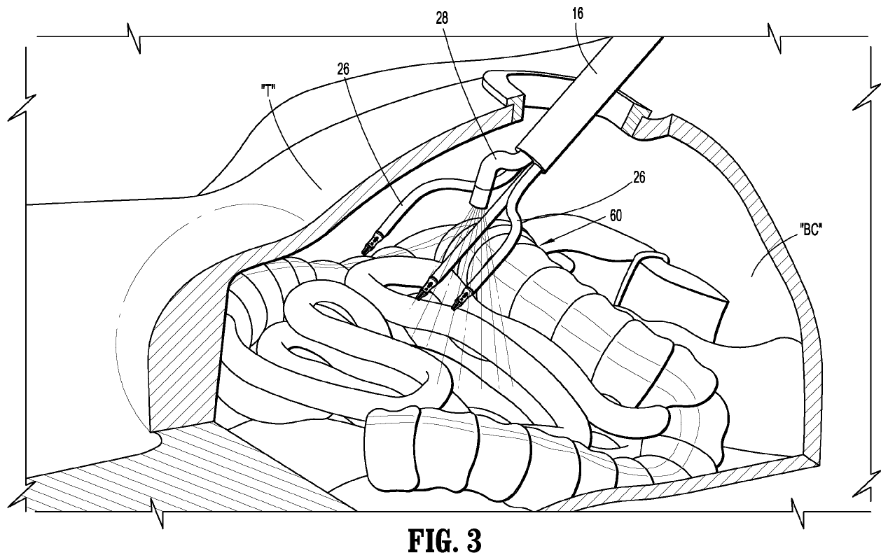

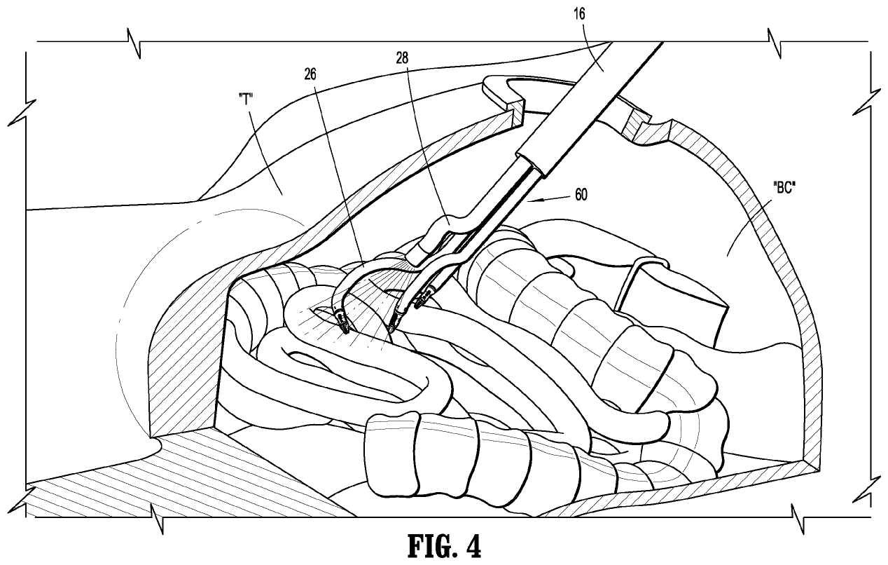

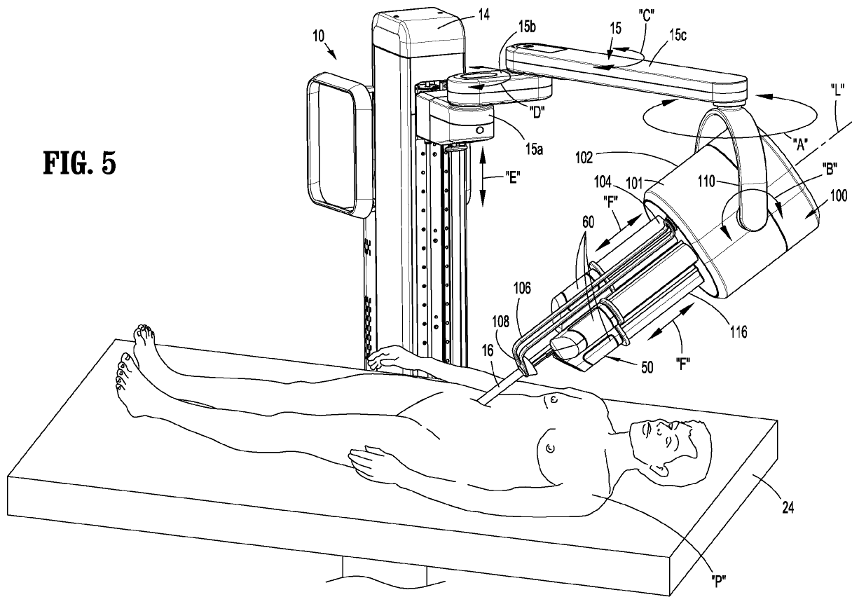

With reference to FIGS. 1-5, a robotic surgical system is shown generally at 10. Robotic surgical system 10 employs various robotic elements to assist the clinician and allow remote operation (or partial remote operation) of surgical instruments 60 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. The instrument cart 14 supports a control drive assembly 100. Control drive assembly 100 includes one or more surgical instrument systems 50 mounted on a control drive unit 101. Control drive unit 101 is movable relative to cart 14 and houses an instrument drive assembly 103 for manipulating the surgical instrument systems 50 and/or independent surgical instruments 60 thereof with the assistance of, for example one or more computing devices or controllers. Although only four surgical instruments 60 are shown, surgical instrument system 50 can include any number and/or type of surgical instruments. The surgical instruments 60 can include, for example, graspers or forceps 26, which may be electrosurgical, an endoscope 28, and/or any other suitable instrument that can be driven by one or more associated tool drives (not shown) of instrument drive assembly 103. For example, besides graspers 26 and endoscope 28, the one or more surgical instruments 60 can include dexterous tools, such as grippers, needle drivers, staplers, dissectors, cutters, hooks, graspers, scissors, coagulators, irrigators, suction devices, that are used for performing a surgical procedure.

Surgical instrument system 50 includes an insertion tube 16 defining a plurality of separate conduits, channels or lumens 16a therethrough that are configured to receive, for instance, the surgical instruments 60 for accessing a body cavity “BC” of a patient “P.” In other aspects, the insertion tube 16 may define a single conduit, channel or lumen therethrough that is configured to receive, for instance, the surgical instruments 60 for accessing a body cavity “BC” of a patient “P.” In particular, the insertion tube 16 can be inserted through an incision “I” and/or access devices 17a, 17b (e.g., a surgical portal, which may include or more seals to facilitate scaled insertion through tissue “T” of the patient “P”) and into the body cavity “BC” of the patient “P”). With insertion tube 16 positioned in the patient “P.” the surgical instruments 60 can be advanced through insertion tube 16 into the body cavity “BC” of the patient “P.” Further, the workstation 12 includes an input device 22 in communication with control drive unit 101 for use by a clinician to control the insertion tube 16 and the various surgical instrument systems 50 (and surgical instruments 60 thereof) via the instrument drive assembly 103 for performing surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like.

The workstation 12 can further include computing devices and/or controllers such as a master processor circuit 22a in communication with the input device 22 for receiving the input signals and generating control signals for controlling the robotic surgical system 10, which can be transmitted to the instrument cart 14 via an interface cable 22b. In some cases, transmission can be wireless and interface cable 22b may not be present. The input device 22 can include right and left-hand controls (not shown) and/or foot pedals (not shown), which are moved/operated to produce input signals at the input device 22 and/or to control robotic surgical system 10. The instrument cart 14 can include a slave processor circuit 20a that receives and the control signals from the master processor circuit 22a and produces slave control signals operable to control the various surgical instrument systems 50 (and surgical instruments 60 thereof) during a surgical procedure. The workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

Control drive unit 101 of control drive assembly 100 includes a housing 102 that supports instrument drive assembly 103, a motor block assembly 104 of instrument drive assembly 103, and a support arm 106 that extends distally from housing 102 to a port latch 108 on a distal end of support arm 106 for supporting insertion tube 16.

Housing 102 of control drive unit 101 includes a mounting clevis 110 that movably (e.g., pivotably—yaw and/or pitch, as indicated by arrows “A” and “B,” respectively) secures control drive unit 101 to a setup arm assembly 15 of robotic surgical system 10, namely, a distal setup arm 15c, of setup arm assembly 15. Distal setup arm 15c is pivotably cantilevered from an intermediate set up arm 15b, as indicated by arrows “C,” which is pivotally cantilevered from a proximal set up arm 15a, as indicated by arrows “D.” Proximal set up arm 15a is secured to instrument cart 14, and axially movable relative thereto, as indicated by arrows “E.”

Motor block assembly 104 of instrument drive assembly 103 includes a plurality of motor block modules 116, each of which is independently axially movable relative to the other motor block modules 116 along a longitudinal axis “L” defined through control drive unit 101, as indicated by arrows “F.”

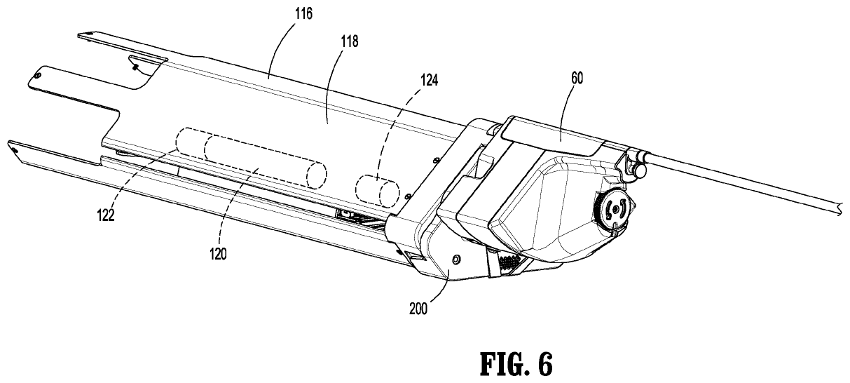

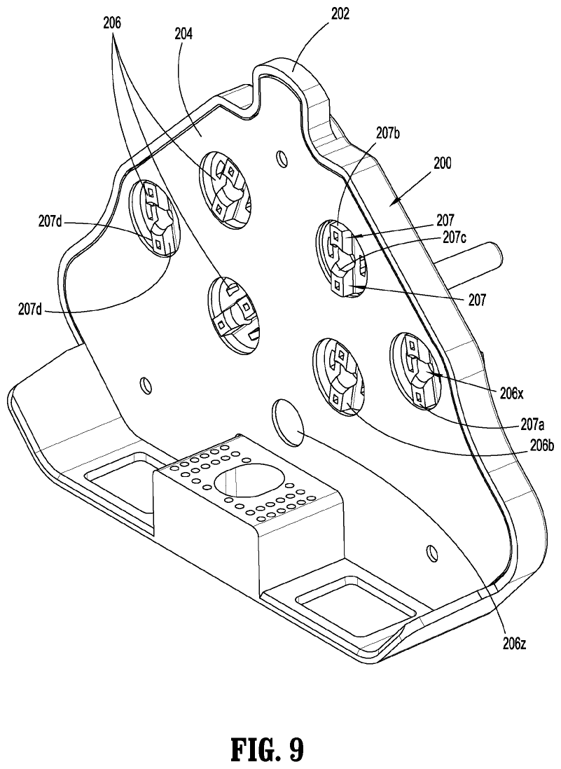

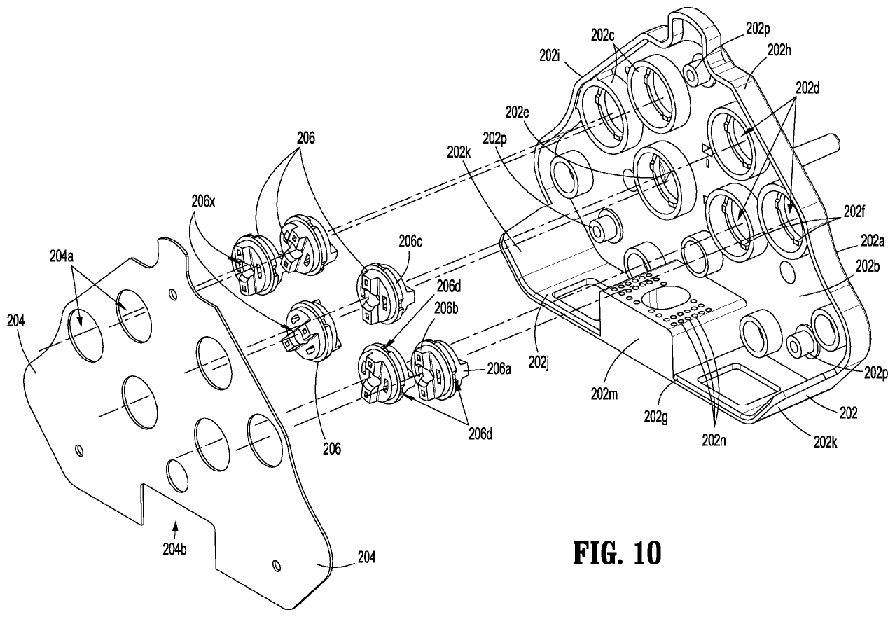

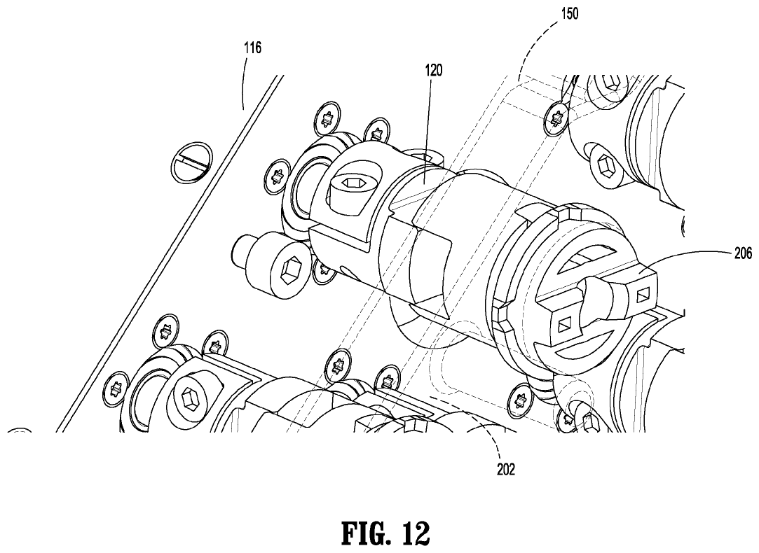

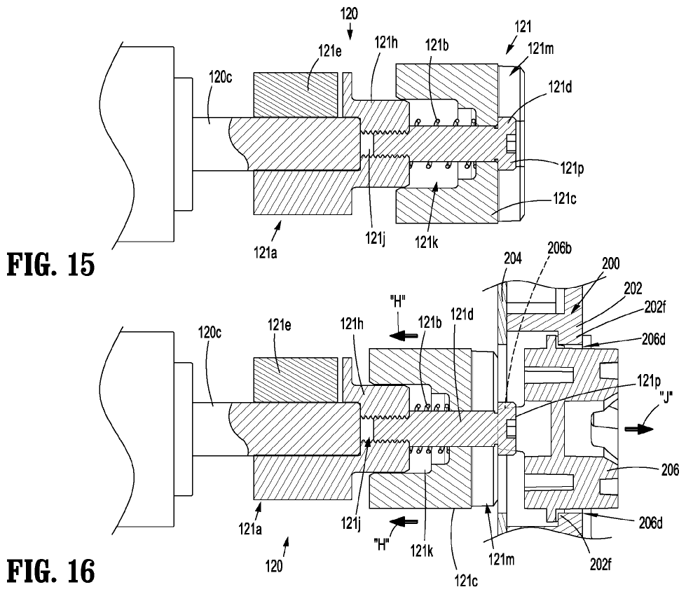

Turning now to FIGS. 6-19, each motor block module 116 of motor block assembly 104 includes a module casing 118 that houses any number of motor assemblies 120, driver boards 122 (e.g., a controller or printed circuit board), and cooling fans 124 (for cooling motor assemblies 120 and/or driver boards 122) therein for efficiently and effectively imparting rotational driving force to surgical instruments 60 attached to the respective motor block module 116 via a sterile adapter assembly 200. Motor assemblies 120, driver boards 122, and/or cooling fans 124 may be disposed in electrical communication with workstation 12 of robotic surgical system 10 to enable a clinician to operate the motor block modules 116 of motor block assembly 104. Each motor block module 116 extends to a distal end cap 150 that encloses module casing 118 and supports motor assemblies 120.

Distal end cap 150 of motor block module 116 has a triangular configuration including a first side 150a and a second side 150b that are connected by a base 150c on a first end of first and second sides 150a, 150b and an apex 150d on second end of first and second sides 150a, 150b. Apex 150d of distal end cap 150 includes an arched tab 150e supported by spaced-apart legs 150f that extend distally from a distal end face 150g of distal end cap 150. Distal end cap 150 defines a locking groove 150h with a sloped surface 150i in distal end face 150g that extends between legs 150f and through apex 150d proximal to arched tab 150c. Locking groove 150h is configured to secure sterile adapter assembly 200 to distal end cap 150. Distal end cap 150 further includes a guidepost 150j that extends distally from distal end face 150g thereof to facilitate engagement with sterile adapter assembly 200. Distal end cap 150 further includes a cooling channel 150k through base 150c of distal end cap 150 to enable air flow from motor block module 116. Base 150c further includes shoulder cutouts 150m adjacent first and second sides 150a, 150b that receive sterile adapter assembly 200 to facilitate securement of sterile adapter assembly 200 to distal end cap 150. Distal end cap 150 also defines motor coupler openings 150n therethrough for supporting motor couplers of motor assemblies 120.

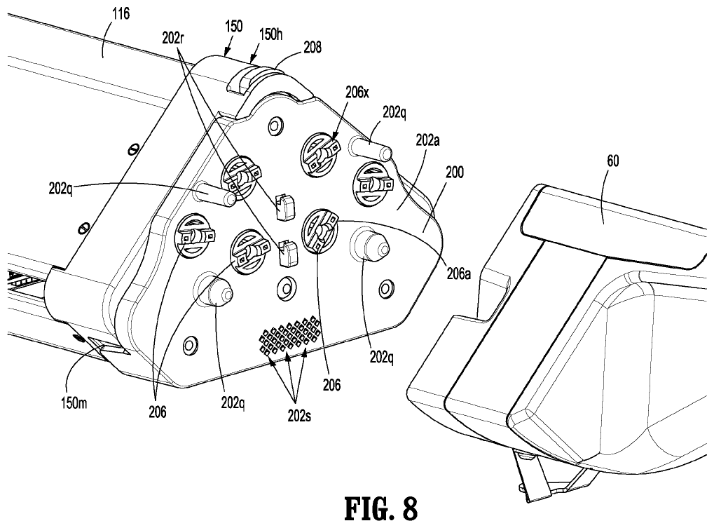

Sterile adapter assembly 200 of robotic surgical system 10 has a triangular configuration that corresponds to distal end cap 150 of motor block module 116. Sterile adapter assembly 200 includes an adapter body 202 and an adapter plate 204 that support adapter couplers 206 therebetween. Sterile adapter assembly 200 further includes a tongue 208 that forms an apex of sterile adapter assembly 200. Tongue 208 is configured to be received by locking groove 150h of distal end cap 150 of motor block module 116 to secure sterile adapter assembly 200 to distal end cap 150 of motor block module 116 (see FIG. 8).

Adapter body 202 of sterile adapter assembly 200 has a distal end face 202a and a proximal end face 202b. Adapter body 202 includes spaced-apart coupler support rings 202c that extend from proximal end face 202b and define openings 202d therethrough for receiving distal portions of adapter couplers 206. Each coupler support ring 202c includes an annular lip 202e that extends proximally from opening 202d and is disposed radially inward from coupler support ring 202c. Annular lip 202e supports anti-rotation bumps 202f at spaced locations about a circumference of annular lip 202c. Adapter body 202 also includes a base wall 202g that extends proximally from first and second sidewalls 202h, 202i of adapter body 202 to define a support ledge 202j. Base wall 202g includes shoulders 202k on opposite sides of support ledge 202j. Support ledge 202j includes a guide rib 202m with air vents 202n defined therein. Adapter body 202 also includes a plurality of fastening posts 202p that receive fasteners for securing adapter plate 204 to adapter body 202. Distal end face 202a of adapter body 202 includes a plurality of guide posts 202q that facilitate aligned engagement with surgical instrument 60. Distal end face 202a of adapter body 202 also includes latching tabs 202r extending therefrom for enabling secured connection between sterile adapter assembly 200 and surgical instrument 60. Additionally, distal end face 202a defines air vents 202s that are disposed in fluid communication with air vents 202n for facilitating air flow therethrough.

Adapter plate 204 of sterile adapter assembly 200 conforms to adapter body 202 of sterile adapter assembly 200 and is supported on support ledge 202j of adapter body 202. Adapter plate 204 defines coupler apertures 204a that align with openings 202d of adapter body 202 for supporting a proximal end portion of adapter couplers 206. Adapter plate 204 further defines a cutout 204b that receives and is supported on guide rib 202m of adapter body 202 and a guidepost passage 206z that receives the guidepost 150j of distal end cap 150.

Each adapter coupler 206 of sterile adapter assembly 200 includes a distal cleat 206a and a proximal cleat 206b that are disposed transverse (e.g., orthogonal) to one another and are coupled together by a central plate 206c. Distal and proximal cleats 206a, 206b define central bores 206x therein. Cleats 206a, 206b each have teeth 207 on opposed sides of central bore 206x. Each tooth 207 has a top surface 207a, a rear surface 207b, a front surface 207c, and a pair of side surfaces 207d that are angled relative to top surface 207a. In particular, side surfaces 207d are inclined such that side surfaces 207d taper to top surface 207a at a predetermined angle to help eliminate long travel and misalignment, to help case component assembly, to help limit backlash, and to help impart axial drive force through adapter coupler 206. The predetermined angle may range, for example, between about three degrees to about 7 degrees, although any suitable angle may be provided. Central plate 206c defines a plurality of anti-rotation recesses 206d that are defined within an outer surface of central plate 206c at spaced-apart locations about a circumference of central plate 206c. Anti-rotation recesses 206d are configured to engage anti-rotation bumps 202f of adapter body 202 to selectively prevent adapter coupler 206 from rotating.

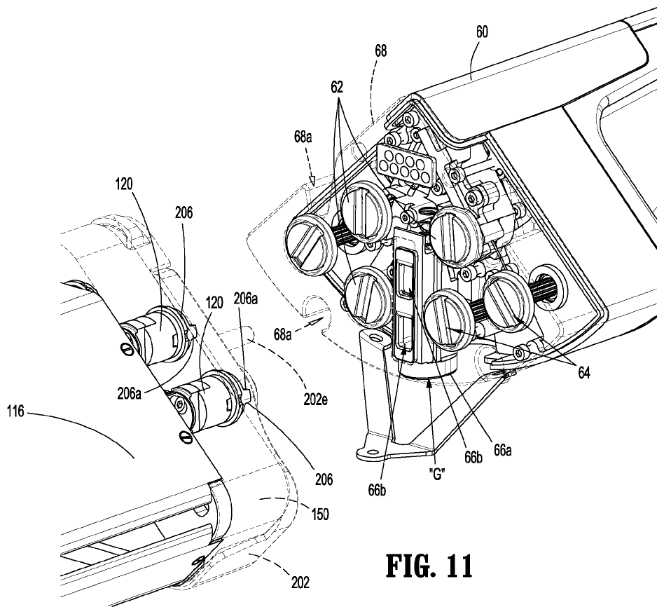

As seen in FIG. 11, surgical instrument 60 of robotic surgical system 10 includes instrument couplers 62 that are configured to engage with adapter couplers 206 of sterile adapter assembly 200. Instrument couplers 62 can be spring biased and define an engagement slot 64 that is configured to receive distal cleat 206a of adapter coupler 206 of sterile adapter assembly 200. Surgical instrument 60 further includes a movable latching mechanism 66 that is selectively engageable with latching tabs 202r of sterile adapter assembly 200. Movable latching mechanism 66 includes a button 66a that is depressible to move latch openings 66b axially, as indicated by arrow “G,” relative to latching tabs 202r to selectively release surgical instrument 60 from sterile adapter assembly 200. Surgical instrument 60 further includes an instrument housing 68 defining a plurality of guide post channels 68a that are configured to receive guide posts 202q of sterile adapter assembly 200 to facilitate alignment between surgical instrument 60 and sterile adapter assembly 200 when surgical instrument 60 is secured to sterile adapter assembly 200.

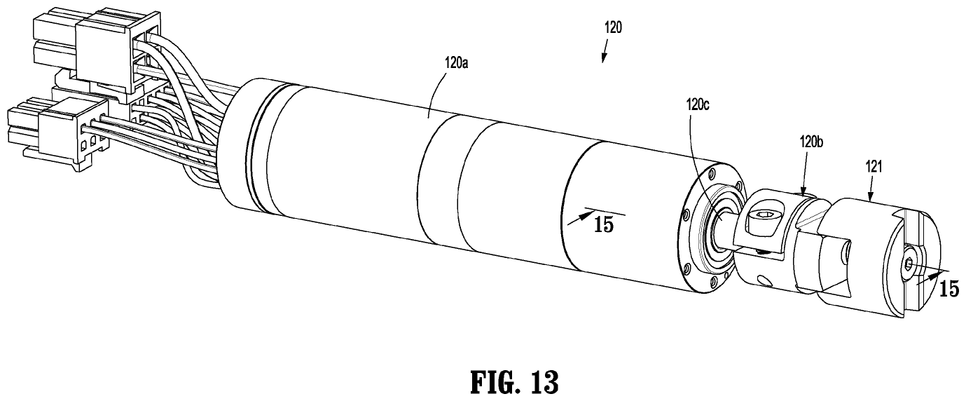

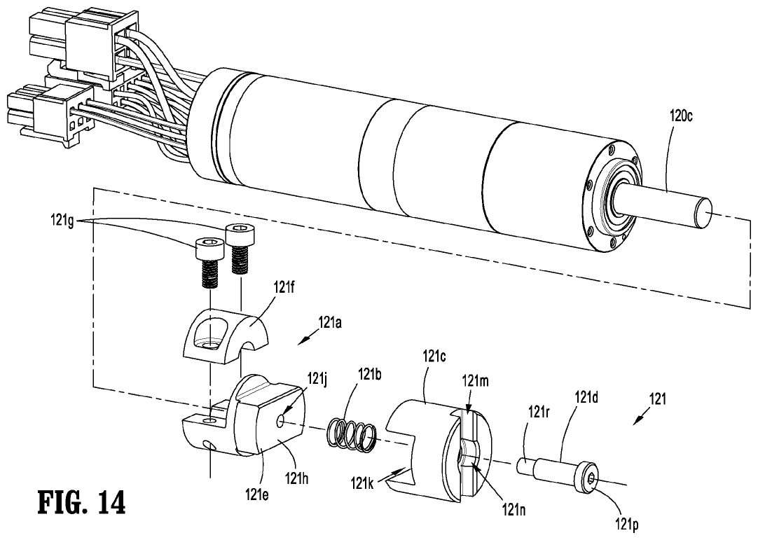

With reference to FIGS. 13 and 14, each motor assembly 120 of motor block module 116 includes a motor 120a having electrical connectors 120b on a proximal end portion thereof for electrically coupling control drive assembly 100 and a shaft assembly 120b extending from a distal end portion of motor 120a. Shaft assembly 120b of motor assembly 120 includes a rotatable shaft 120c and a motor coupler assembly 121 secured to rotatable shaft 120c. Motor coupler assembly 121 of shaft assembly 120b includes a collar assembly 121a, a spring 121b, a motor coupler 121c, and a shoulder fastener 121d that secures motor coupler 121c and spring 121b to collar assembly 121a. Collar assembly 121a of motor coupler assembly 121 secures to a distal portion of rotatable shaft 120c. Collar assembly 121a includes a collar body 121e, a collar nut 121f, and fasteners 121g that secure the collar nut 121f to collar body 121e about rotatable shaft 120c so that collar assembly 121a rotates with rotatable shaft 120c. Collar body 121e of collar assembly 121a includes a distal drive cleat 121h that defines a threaded opening 121j. Motor coupler 121c of motor coupler assembly 121 includes a cleat channel 121k defined transversely therethrough on a proximal end portion thereof and a drive channel 121m on a distal end portion of motor coupler 121c that is transverse to cleat channel 121k. Motor coupler 121c further defines a central bore 121n therethrough that receives shoulder fastener 121d therethrough. Shoulder fastener 121d defines has a drive head 121p, a spring shaft 121q that extends from drive head 121p and supports spring 121b, and a threaded tip 121r that threadedly couples to threaded opening 121j of collar body 121c. Shoulder fastener 121d secures spring 121b and motor coupler 121 to collar body 121e such that motor coupler 121c is positioned to axially float along shoulder fastener 121d relative to collar body 121e on a distal end portion of rotatable shaft 120 of motor 120a.

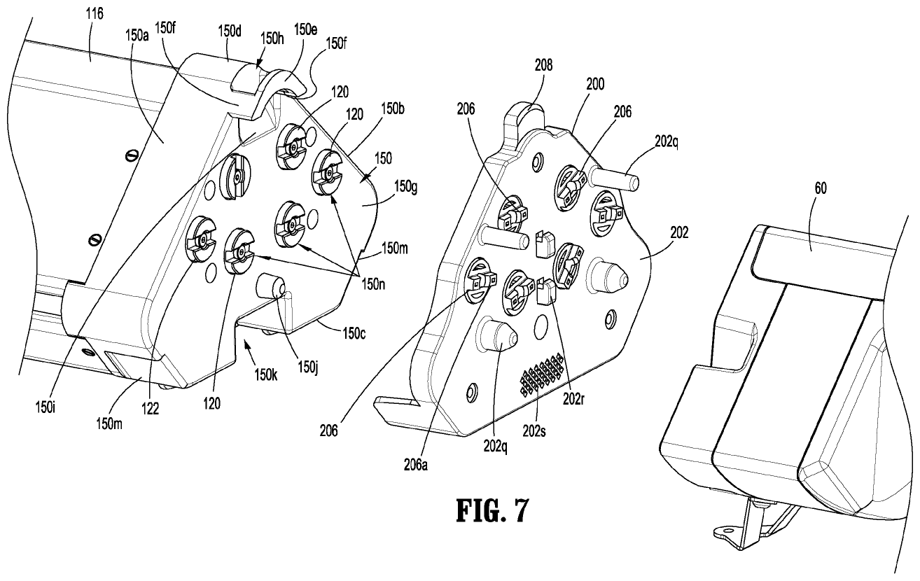

Turning now to FIGS. 7, 8, 11, and 15-19, to secure sterile adapter assembly 200 to motor block module 116, sterile adapter assembly 200 is aligned with distal end cap 150 of motor block module 116 so that tongue 208 of sterile adapter assembly 200 cams into locking groove 150h of distal end cap 150. As tongue 208 slides along locking groove 150h, guide post 150j of distal end cap 150 slides into guide post passage 206z of sterile adapter assembly 200 as guide rib 202m of sterile adapter assembly 200 slides into cooling channel 150k of distal end cap 150. With sterile adapter assembly 200 mounted to distal end cap 150 (FIG. 8), drive head 121p of shoulder fastener 121d engages with central bore 206x of proximal cleat 206b of adapter coupler 206.

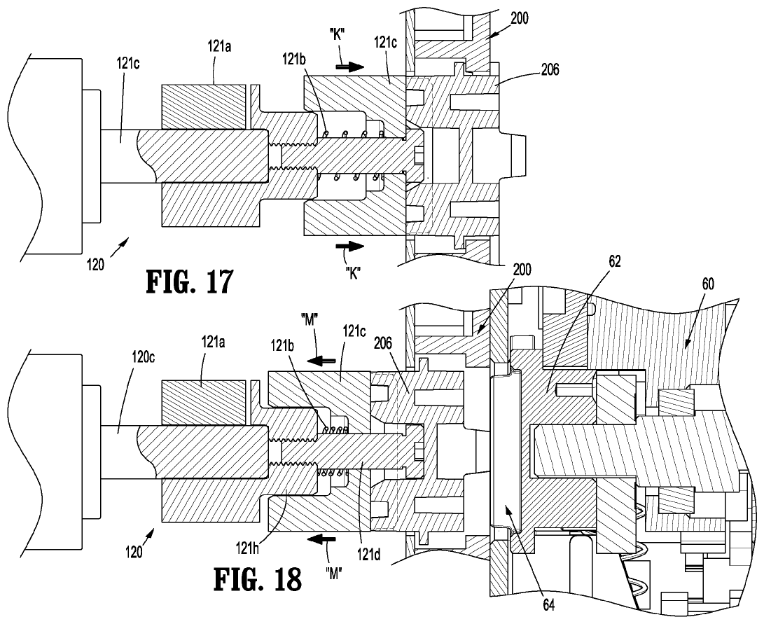

With sterile adapter assembly 200 mounted to distal end cap 150 (FIG. 8), depending on the rotational positions of motor couplers 121c of motor assemblies 120, one or more motor couplers 121c may be perfectly rotationally aligned with one or more adapters couplers 206 of sterile adapter assembly 200 so that drive channels 121m of such motor couplers 121c receive the respective proximal cleats 206b of such adapter couplers 206 to interlock such motor and adapter couplers 121c, 206 (FIG. 17). However, in many instances, one or more of the motor couplers 121c may be rotationally misaligned with respective adapters couplers 206 so that drive channels 121m of such motor couplers 121c and proximal cleats 206b of such adapter couplers 206 do not immediately interlock (FIG. 16).

In particular, when motor couplers 121c of motor assemblies 120 are rotationally misaligned with adapters couplers 206 of sterile adapter assembly 200, proximal cleats 206b of such adapter couplers 206 urge the respective motor couplers 121c proximally, as indicated by arrows “H,” under a spring bias force of the respective spring 121b, and relative to the respective collar body 121e, such that the distal drive cleat 121h of the respective collar body 121e extends farther into cleat channel 121k of such motor coupler 121c as such motor coupler 121c and respective collar body 121e approximate one another. Simultaneously, such motor coupler 121c of such motor assembly 120 applies a distal force, through spring 121b, to the respective adapter coupler 206, driving such adapter coupler 206 distally relative to the respective adapter body 202 of adapter assembly 200, as indicated by arrow “J.”

In certain instances, anti-rotation bumps 202f of adapter body 202 will be perfectly rotationally positioned to receive anti-rotation recesses 206d of a respective adapter coupler 206 to prevent such adapter coupler 206 from rotating with a respective motor coupler 121c as such motor coupler 121c is rotated. In other instances, when anti-rotation bumps 202f are not aligned with anti-rotation recesses 206d, frictional engagement (e.g., high friction) between a distal portion of motor coupler 121c and a proximal portion of adapter coupler 206 enables adapter coupler 206 to rotate relative to adapter body 202 with a rotation of the respective motor coupler 121c. Continued rotation of adapter coupler 206 causes anti-rotation bumps 202f of adapter body 202 to rotationally align with anti-rotation recesses 206d of adapter coupler 206 so that anti-rotation bumps 202f of adapter body 202 interlock with anti-rotation recesses 206d of adapter coupler 206. This prevents adapter coupler 206 from rotating with the respective motor coupler 121c. Such motor coupler 121c can then continue to rotate relative to the respective adapter coupler 206 until drive channel 121m of such motor coupler 121c and proximal cleat 206b of such adapter coupler 206 are aligned. Once aligned, spring 121b can urge such motor coupler 121c distally, as indicated by arrows “K,” so that drive channel 121m of such motor coupler 121c and proximal cleat 206b of such adapter coupler 206 can interlock (FIG. 17).

In still further instances, frictional engagement between a distal portion of a motor coupler 121c and a proximal portion of a respective adapter coupler 206 may be insignificant (e.g., low friction or nonexistent) such that the respective motor coupler 121c may rotate relative to the respective adapter coupler 206 without anti-rotation bumps 202f of adapter body 202 interlocked with anti-rotation recesses 206d of such adapter coupler 206. Like the above, such motor coupler 121c can continue to rotate relative to such adapter coupler 206 until drive channel 121m of such motor coupler 121c and proximal cleat 206b of such adapter coupler 206 are aligned. Once aligned, spring 121b can urge such motor coupler 121c distally, as indicated by arrows “K,” so that drive channel 121m of such motor coupler 121c and proximal cleat 206b of such adapter coupler 206 can interlock (FIG. 17).

Once sterile adapter assembly 200 is mounted to distal end cap 150, and all motor assemblies 120 of distal end cap 150 are aligned and interlocked with adapter couplers 206 of sterile adapter assembly 200, surgical instrument 60 can then be attached to sterile adapter assembly 200.

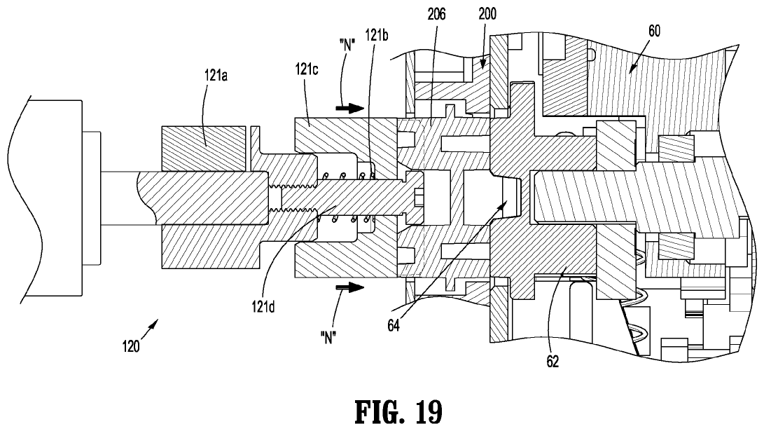

To secure surgical instrument 60 to sterile adapter assembly 200, guideposts 202q of sterile adapter assembly 200 are inserted into guidepost channels 68a of instrument housing 68, and latching openings 66b of latch mechanism 66 receive latching tabs 202r. With surgical instrument 60 secured to sterile adapter assembly 200, depending on the rotational positions of adapter couplers 206, one or more adapter couplers 206 may be perfectly rotationally aligned with respective instrument couplers 62 so that engagement slots 64 of such instrument couplers 62 receive distal cleats 206a of respective adapter couplers 206 for immediately interlocking such adapter couplers 206 and instrument couplers 62 (FIG. 19). Securement of surgical instrument 60 to sterile adapter assembly 200 causes such adapter couplers 206 to move proximally so that anti-rotation bumps and recesses to separate for allowing adapter couplers 206 to rotate with motor couplers 121c.

However, in many instances, one or more adapter couplers 206 may be rotationally misaligned with respective instrument couplers 62 so that engagement slots 64 of such instrument couplers 62 and distal cleats 206a of such adapter couplers 206 do not immediately interlock (FIG. 18). With such misalignment, securement of surgical instrument 60 to sterile adapter assembly 200 imparts a proximal force through adapter couplers 206 that separates anti-rotation bumps 202f from anti-rotation recesses 206d so adapter couplers 206 move proximally with motor couplers 121c, as indicated by arrows “M,” compressing spring 121b of the respective motor assemblies 120, so that respective adapter couplers 206 and motor couplers 121c can rotate together. Then, such motor couplers 121c and adapter couplers 206 are rotated together relative to respective instrument couplers 62 until distal cleats 206a of such adapter couplers 206 align with engagement slots 64 of respective instrument couplers 62 for enabling springs 121b to distally advance respective motor couplers 121c and adapter couplers 206 into interlocked engagement with respective instrument couplers 62, as indicated by arrows “N.” Adapter couplers 206 can frictionally engage with instrument couplers 62, similar to the frictional engagement detailed above with respect to motor couplers 121c and adapter couplers 206, so that such adapter couplers 206 and instrument couplers 62 can rotate together and/or independently (e.g., adapter coupler 206 can rotate relative to instrument coupler 62). Indeed, instrument housing 68 and instrument couplers 62 can also include anti-rotation bumps and/or recesses (not explicitly shown) to enable adapter couplers 206 to rotate relative to respective instrument couplers 62 and to selectively prevent such instrument couplers 62 from rotating.

Once all instrument couplers 62 are aligned and interlocked with respective adapter couplers 206, rotation of respective motor couplers 121c enables the end effector of surgical instrument 60 to operate (e.g., rotate, articulate, grasp, etc.). Surgical instrument 60 can then be removed as desired, such as for an instrument exchange, by actuating latch mechanism 66 of surgical instrument. Sterile adapter assembly 200 can then be removed as desired or secured to the same or different surgical instrument using the techniques described herein, as desired.

The disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing devices (and/or servers) can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” “in other aspects” or the like may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to all such states. Reference to a program may encompass the actual instructions and/or the intent of those instructions.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A robotic surgical system, comprising:
a motor block module supporting a motor assembly, the motor assembly including a motor having a shaft assembly, the shaft assembly including a rotatable shaft and a motor coupler assembly secured to the rotatable shaft, the motor coupler assembly including a motor coupler and a spring engaged with the motor coupler; and
a sterile adapter assembly mounted to the motor block module and supporting an adapter coupler that is positioned to engage with the motor coupler, the adapter coupler positioned to impart axial force onto the motor coupler sufficient to compress the spring when the adapter coupler and the motor coupler are rotationally misaligned, the motor coupler being rotatable relative to the adapter coupler to rotationally align the motor coupler and the adapter coupler, the spring positioned to move the motor coupler toward the adapter coupler when the motor coupler and the adapter coupler are rotationally aligned such that the adapter coupler and the motor coupler can rotate together.

2. The robotic surgical system of claim 1, wherein the motor coupler assembly includes a collar assembly that secures to the rotatable shaft.

3. The robotic surgical system of claim 2, wherein the spring extends between the motor coupler and the collar assembly.

4. The robotic surgical system of claim 3, wherein the spring is supported on a fastener that secures the motor coupler to the collar assembly.

5. The robotic surgical system of claim 4, wherein the motor coupler is axially movable along the fastener and relative to the collar assembly as the spring moves between a compressed position and an expanded position.

6. The robotic surgical system of claim 1, wherein the sterile adapter assembly includes an adapter body and an adapter plate that support the adapter coupler therebetween.

7. The robotic surgical system of claim 6, wherein the adapter body includes an anti-rotation feature that selectively engages with an anti-rotation feature of the adapter coupler to prevent the adapter coupler from rotating when the motor coupler and the adapter coupler are misaligned.

8. The robotic surgical system of claim 1, wherein the sterile adapter assembly includes a tongue, and the motor block module supports a distal end cap, the distal end cap defining a locking groove that is positioned to receive the tongue of the sterile adapter assembly to secure the sterile adapter assembly to the distal end cap.

9. The robotic surgical system of claim 1, wherein the motor coupler includes a drive channel on a distal end portion thereof and the adapter coupler includes a proximal cleat that is positioned to be received within the drive channel of the motor coupler when the adapter coupler and the motor coupler are rotationally aligned.

10. The robotic surgical system of claim 9, wherein the adapter coupler includes a distal cleat that is configured to engage with an instrument coupler of a surgical instrument.

11. A surgical system, comprising:
a motor block module supporting a motor assembly, the motor assembly including a motor having a shaft assembly, the shaft assembly including a shaft and a motor coupler assembly secured to the shaft, the motor coupler assembly including a motor coupler and a spring engaged with the motor coupler;
an adapter assembly mounted to the motor block module and supporting an adapter coupler that is positioned to engage with the motor coupler, the adapter coupler positioned to impart force onto the motor coupler sufficient to compress the spring when the adapter coupler and the motor coupler are misaligned, the motor coupler being movable relative to the adapter coupler to align the motor coupler and the adapter coupler, the spring positioned to move the motor coupler toward the adapter coupler when the motor coupler and the adapter coupler are aligned such that the adapter coupler and the motor coupler can move together; and
a surgical instrument securable to the adapter assembly.

12. The surgical system of claim 11, wherein the motor coupler assembly includes a collar assembly that secures to the shaft.

13. The surgical system of claim 12, wherein the spring extends between the motor coupler and the collar assembly.

14. The surgical system of claim 13, wherein the spring is supported on a fastener that secures the motor coupler to the collar assembly.

15. The surgical system of claim 14, wherein the motor coupler is axially movable along the fastener and relative to the collar assembly as the spring moves between a compressed position and an expanded position.

16. The surgical system of claim 11, wherein the adapter assembly includes an adapter body and an adapter plate that support the adapter coupler therebetween.

17. The surgical system of claim 16, wherein the adapter body includes an anti-rotation feature that selectively engages with an anti-rotation feature of the adapter coupler to prevent the adapter coupler from rotating when the motor coupler and the adapter coupler are misaligned.

18. The surgical system of claim 11, wherein the adapter assembly includes a tongue, and the motor block module supports a distal end cap, the distal end cap defining a locking groove that is positioned to receive the tongue of the adapter assembly to secure the adapter assembly to the distal end cap.

19. The surgical system of claim 11, wherein the motor coupler includes a drive channel on a distal end portion thereof and the adapter coupler includes a proximal cleat that is positioned to be received within the drive channel of the motor coupler when the adapter coupler and the motor coupler are aligned.

20. A robotic surgical system, comprising:
a motor block module supporting a motor assembly, the motor assembly including a motor having a shaft assembly, the shaft assembly including a shaft and a motor coupler secured to the shaft;
an adapter assembly mountable to the motor block module and supporting an adapter coupler that is positioned to engage with the motor coupler, the motor coupler rotatable relative to the adapter coupler when the motor coupler and the adapter coupler are misaligned and rotatable with the adapter coupler when the motor coupler and the adapter coupler are aligned; and
a surgical instrument securable to the adapter assembly, the surgical instrument including an instrument coupler that is positioned to engage with the adapter coupler, the adapter coupler positioned to rotate relative to the instrument coupler when the instrument coupler and the adapter coupler are rotationally misaligned to rotationally align the adapter coupler with the instrument coupler.
